(19) United States
(12) Patent Application Publication
(10) Pub. No.: US 2025/0213862 A1
(43) Pub. Date: Jul. 3, 2025

(54) PERSONALIZED BRAIN STIMULATION DEVICE

(71) Applicant: LEE SOL CO., LTD., Seoul (KR)

(72) Inventors: Seung Woo LEE, Seoul (KR); Gusung KWON, Seoul (KR)

(21) Appl. No.: 18/746,086

(22) Filed: Jun. 18, 2024

(30) Foreign Application Priority Data
Dec. 28, 2023 (KR) 10-2023-0194661

(51) Int. Cl. A61N 1/36; A61N 1/04

(52) U.S. Cl. CPC A61N 1/36025; A61N 1/0476; A61N 1/36031; A61N 1/36034

(57) ABSTRACT

A personalized brain stimulation device includes: a sensor unit measuring biological information of an object; a control unit determining, based on the biological information of the object measured by the sensor unit, that the state of the object corresponds to a first state among a predetermined plurality of depression-related states; and a stimulation unit delivering a stimulus, which varies according to the determined first state, to the brain of the object to entrain synchronized oscillation in multiple regions of the brain of the object, wherein the stimulus is a first stimulus to entrain gamma oscillation synchronized in multiple regions of the brain, a second stimulus to entrain any one of delta, theta, alpha, and beta oscillation synchronized in multiple regions of the brain, or a third stimulus that combines the first stimulus and the second stimulus, wherein the first, second, and third stimulus are an transcranial alternating current stimulation.

### CROSS-REFERENCE TO RELATED APPLICATIONS

This application claims the priority to Korean Patent Application No. 10-2023-0194661 filed on Dec. 28, 2023, in the Korean Intellectual Property Office, the disclosure of which is incorporated herein by reference.

### BACKGROUND

### Field

The present invention relates to a personalized brain stimulation device, and more specifically, to a personalized brain stimulation device capable of personalizing brain stimulation based on the patient's biological information, determining the patient's depression state, and delivering tACS (transcranial Alternating Current Stimulation) based composite stimulation to the patient's brain to improve the patient's depression state.

### Description of the Related Art

Recently, there has been a shift towards treating diseases with electronic drugs instead of oral medications. Electronic drugs are medical devices that produce new therapeutic effects by acting on brain nerves with energy such as electricity, magnetic fields, or ultrasound, rather than inducing symptom relief through biochemical actions within the body like pharmaceuticals. They may be seen as a new field of miniaturized medical devices related to brain nerve function regulation.

Further, electronic drugs can treat diseases with electrical stimulation instead of directly administering drugs to the patient's body, like oral medications or injections. This ability to selectively stimulate only the specific area requiring treatment makes them safer for the human body. In other words, patients who are averse to taking medications can reduce side effects with electronic drug treatments and can achieve therapeutic synergy when combined with medications.

In Korea, an electronic drug for depression that normalizes the frontal lobe function, which is the cause of depression, has been released. Methods of stimulation for electronic drugs to treat depression include tACS (transcranial Alternating Current Stimulation), tDCS (transcranial Direct Current Stimulation), DBS (Deep Brain Stimulation), tMS (transcranial Magnetic Stimulation), and ECT (Electrconvulsive Therapy).

Firstly, tACS is a method of attaching electrodes and delivering a small current of less than 1 mA to the skull. It is used for non-pharmacological treatments to improve symptoms such as anxiety, depression, insomnia, stress, headaches, and various types of pain. It is effective in regulating microglia, safe due to the use of microcurrents, and free of side effects, allowing for long-term treatment. Moreover, as a cutting-edge treatment method compatible with existing chemical therapies that promote and/or inhibit hormone secretion, it additionally enables sleep induction and quality improvement through the stable maintenance of DMN (default mode network) in the brain, and can improve sleep induction and quality through the enhancement of hormones (serotonin, melatonin, GABA, etc.). It can stimulate brain tissues to restore neurochemical balance before stress. The stimulation energy of tACS may be AC (current flow), and the stimulation form may be pulse or sinusoidal. The mechanism may be brain entrainment by current, and a feature is high patient convenience due to verified safety (0.5 mA or 500 μA). However, due to skin impedance, which is a value that obstructs the flow of current when voltage is applied in a circuit, there is no pain, but the stimulation delivery is weak, making it difficult to perceive the stimulation.

tDCS is a method of attaching electrodes to the head and stimulating the cerebral cortex's neurons with a weak direct current. It is a non-invasive brain stimulation method for recovering from aftereffects of brain damage. By controlling the activation state of brain nerves through electrical stimulation, it can help improve brain functions. The stimulation energy of tDCS may be DC (electric field), and the stimulation form may be direct current (DC). The mechanism is to maintain balance by the activation (+)/inhibition (−) charge of the electrode position. The feature is the simplicity of application (2 mA). However, there is a downside that the difference between real stimulation and sham stimulation is not significant, making the treatment effect for depression low and it is a simple procedure with non-invasive stimulation but poses a burn risk at the electrode attachment site.

DBS is a method of stimulating the activity of nerve cells by placing fine needle electrodes in the deep nuclei of the brain. By applying electrical stimulation to the nucleus of a specific brain area, it can interfere with pathological signals that occur in the brain regions, thereby treating and improving symptoms of various diseases including motor disorders. It places fine electrodes in the deep nuclei of the brain and receives the necessary power for activity from a pulse generator implanted in the chest in a manner similar to a pacemaker, allowing for depolarization block, i.e., blocking the neural output of nerve cells located at the electrode site. Further, it can inhibit the synapse, in other word, it can activate axon terminals with synaptic connections to neurons near the electrodes, thereby indirectly controlling the neural output of nerve cells. The stimulation energy of DBS may be direct current (DC), pulse, or near-infrared (NIR). The stimulation form may be pulse or sinusoidal. The mechanism is direct stimulation of the deep brain, and it may be effective with high-frequency stimulation (130 Hz). The feature is a high treatment effect for depression compared to other electronic drug stimulation methods due to high-frequency stimulation. However, an additional surgical procedure is needed to insert the needles into the deep nuclei of the brain.

tMS is a method of non-invasively stimulating neurons in the brain using magnetic energy. It is effective in treating neurological and psychiatric disorders such as Parkinson's syndrome and depression. By generating a strong magnetic field with a conductive electromagnetic coil close to the head, this magnetic field penetrates the skull and stimulates the neurons of the transcranial cortex. Depending on the speed of the magnetic field, it can increase or decrease the activation of the cerebral cortex. For example, for low cortical activation in conditions such as depression, high-frequency stimulation is used, and for high cortical activation in conditions such as anxiety or mania, low-frequency stimulation is used to control activation levels. The stimulation energy of tMS may be edge current by a magnetic field, and the stimulation form may be activation with 10 Hz pulse (5 Hz iTBS) and inhibition with 1 Hz pulse. The mechanism is energy supply by the magnetic field, leading to an increase in gamma band power. The feature is non-invasive stimulation, not requiring a surgical procedure causing wounds. However, due to the large equipment, patient convenience is reduced, and high-frequency electrical stimulation is realistically impossible due to non-invasive stimulation limitations.

ECT uses AC (current flow) as stimulation energy, and the stimulation form may be pulse or sinusoidal. The mechanism is to reset neurons by strong electrical stimulation (20-70 Hz). However, there is an issue that anesthesia is required to apply strong electrical stimulation to reset neurons.

Meanwhile, tACS, one of the electromagnetic stimulation methods, unlike the other electromagnetic stimulation methods mentioned above, enables real-time monitoring of the patient's brain waves (electroencephalogram, EEG) through brain entrainment by current, allowing for the provision of depression biomarkers to the practitioner to check the patient's depression state.

In other words, tACS-based electronic drugs for depression, unlike electronic drugs based on other stimulation methods, are expected to be more effective in treating the patient's depression by allowing for personalized brain stimulation according to the patient's depression state.

However, to provide tACS-based electronic drugs for depression treatment, it is necessary to improve the issue of weak stimulation delivery due to skin impedance in tACS.

### SUMMARY

The problem to be solved by the present invention is to provide a personalized brain stimulation device capable of determining the patient's depression state based on the patient's biological information and delivering tACS (transcranial Alternating Current Stimulation) based composite stimulation to the patient's brain to improve the patient's depression state.

Specifically, the present invention aims to provide a personalized brain stimulation device capable of determining the patient's depression state based on at least one of the patient's biological information such as heart rate variability (HRV) information, brain wave (electroencephalogram, EEG) information, heart rate information, stress information, body composition information, weight information, oxygen saturation information, pulse information, blood pressure information, iris information, voice information, vein information, and electrocardiogram (ECG) information, and delivering tACS (Transcranial alternating current stimulation) based composite stimulation to the patient's brain to improve the patient's depression state.

Further, the present invention aims to provide a personalized brain stimulation device capable of enhancing gamma-level brain waves in the patient's brain through gamma oscillation entrainment when the patient's depression-related state is in a major depressive disorder (MDD) state.

Further, the present invention aims to provide a personalized brain stimulation device capable of reducing gamma-level brain waves in the patient's brain through the entrainment of any one of delta, theta, alpha, and beta oscillations when the patient's depression-related state is in a depressive disorder state due to stress.

Further, the present invention aims to provide a personalized brain stimulation device capable of enhancing gamma-level brain waves when the gamma level is lowest in the patient's brain and reducing gamma-level brain waves when the gamma level is highest in the patient's brain, when the patient's depression-related state is in a bipolar disorder (BD) state.

However, the technical problems to be achieved by the present invention are not limited to the above-mentioned technical problems, and other technical problems not mentioned will be clearly understood by those skilled in the art from the description below.

To achieve the above objectives, a personalized brain stimulation device according to an embodiment of the present invention comprises: a sensor unit that measures the biological information of an object; a control unit that determines the state of the object as corresponding to a first state among a plurality of predetermined depression-related states based on the biological information of the object measured by the sensor unit; and a stimulation unit that delivers stimulation to the object's brain, varying according to the first state determined by the control unit, to entrain oscillations in multiple regions of the object's brain, wherein the stimulation is a first stimulation for entraining gamma oscillations synchronized in multiple regions of the brain, a second stimulation for entraining any one of delta, theta, alpha, and beta oscillations synchronized in multiple regions of the brain, or a third stimulation combining the first and second stimulations, wherein the first, second, and third stimulations are transcranial alternating current stimulation (tACS), wherein the transcranial alternating current stimulation repeatedly switches on and off according to a predetermined first frequency, and transcranial alternating current stimulation may be a first combined stimulus where the signal turned on according to the first frequency is applied as stimulation according to a predefined second frequency.

Additionally, the biological information may include at least one of heart rate variability (HRV) information, electroencephalogram (EEG) information, heart rate information, stress information, body composition information, weight information, oxygen saturation information, pulse information, blood pressure information, iris information, voice information, vein information, and electrocardiogram (ECG) information.

Moreover, the predefined plurality of depressive states may include a depressive disorder state caused by stress, a major depressive disorder (MDD) state, and a bipolar disorder (BD) state.

Further, the stimulation unit delivers the first stimulation to the brain when the control unit determines that the object's first state is a major depressive disorder state, and the delivery of the first stimulation can enhance gamma-level brain waves in the object's brain wave through the entrainment of gamma oscillations.

Additionally, the stimulation unit delivers the second stimulation to the brain when the control unit determines that the object's first state is a depressive disorder state caused by stress, and the delivery of the second stimulation can reduce gamma-level brain waves in the object's brain wave through the entrainment of any one of delta, theta, alpha, and beta oscillations.

Moreover, the stimulation unit delivers the third stimulation to the brain when the control unit determines that the object's first state is a bipolar disorder state, and if the object's first state is a bipolar disorder state and the gamma level in the object's brain wave is the lowest, the first stimulation is delivered to enhance the gamma-level brain waves in the object's brain wave, and if the object's first state is a bipolar disorder state and the gamma level in the object's brain wave is the highest, the second stimulation is delivered to reduce the gamma-level brain waves in the object's brain wave.

Further, the brain wave information is a first brain wave obtained from the brain, and the sensor unit measures a second brain wave of the object in the state where the stimulation is delivered to the brain, and the control unit can determine whether a first response to entrain oscillations in multiple regions of the brain is derived based on the second brain wave of the object measured by the sensor unit.

Additionally, the first frequency is applied to induce the entrainment of oscillations in multiple regions of the brain, and the second frequency is applied to induce membrane action potential and brain oscillation in multiple regions of the brain, and may be higher than the first frequency.

Further, the control unit processes the first signal measured by the sensor unit due to the first combined stimulation as noise, and determines whether the first response is derived based on the second signal excluding the first signal measured by the sensor unit.

Moreover, if the first response according to the first combined stimulation is not derived, the control unit can control the stimulation unit to deliver a second combined stimulation, which is modified in at least one of the first frequency, the second frequency, the output of the stimulation according to the second frequency, the waveform, and the period, to the brain of the object.

Additionally, the first response is related to the entrainment of gamma oscillation or any one of delta, theta, alpha and beta oscillations synchronized in multiple regions of the brain, and the first frequency is a frequency of 30 Hz to 80 Hz to entrain gamma oscillation synchronized in multiple brain regions including the prefrontal cortex (PFC) and hippocampus, a frequency of 14 Hz to 29 Hz to entrain beta oscillations, a frequency of 8 Hz to 13 Hz to entrain alpha oscillations, a frequency of 4 Hz to 7 Hz to entrain theta oscillations, or a frequency of more than 0 Hz and less than 4 Hz to entrain delta oscillations.

Additionally, it is possible to provide the personalized brain stimulation device which may be characterized by ignoring signal interference due to the first combined stimulation based on the magnitude of the first signal being different from the magnitude of the second signal by at least a predetermined value.

According to one embodiment of the present invention, it is possible to provide a personalized brain stimulation device capable of determining a patient's depressive state based on the patient's biological information and delivering complex stimulation based on transcranial alternating current stimulation (tACS) to the patient's brain to improve the patient's depressive state.

Specifically, the present invention aims to provide a personalized brain stimulation device capable of determining a patient's depressive state based on at least one of the patient's biological information such as heart rate variability (HRV) information, electroencephalogram (EEG) information, heart rate information, stress information, body composition information, weight information, oxygen saturation information, pulse information, blood pressure information, iris information, voice information, vein information, and electrocardiogram (ECG) information, and delivering complex stimulation based on transcranial alternating current stimulation (tACS) to the patient's brain to improve the patient's depressive state.

Further, the present invention aims to provide a personalized brain stimulation device capable of enhancing gamma-level brain waves in the patient's brain wave through the entrainment of gamma oscillations when the patient's depressive state is a major depressive disorder (MDD) state.

Additionally, the present invention aims to provide a personalized brain stimulation device capable of reducing gamma-level brain waves in the patient's brain wave through the entrainment of any one of delta, theta, alpha, and beta oscillations when the patient's depressive state is a depressive disorder state caused by stress.

Moreover, the present invention aims to provide a personalized brain stimulation device capable of enhancing gamma-level brain waves when the gamma level is lowest in the patient's brain waves, and reducing gamma-level brain waves when the gamma level is highest in the patient's brain waves, when the patient's depressive state is a bipolar disorder (BD) state.

However, the effects obtainable from the present invention are not limited to the effects mentioned above, and other effects not mentioned will be clearly understood by those skilled in the art from the descriptions below.

### BRIEF DESCRIPTION OF THE DRAWINGS

FIG. 1 is a block diagram of a brain stimulation system according to one embodiment of the present invention.

FIG. 2 is a diagram illustrating an example of a brain stimulation device.

FIG. 3 is a diagram for explaining a plurality of predetermined depressive states.

FIG. 4 is a diagram for explaining the EEG signal-based closed-loop neuro-feedback tACS stimulation method proposed by the present invention.

FIG. 5 is a block diagram of a real-time tACS-EEG neurofeedback algorithm according to one embodiment of the present invention.

FIG. 6 is a diagram illustrating the clinical protocol of a clinical trial.

### DETAILED DESCRIPTION OF THE PREFERRED EMBODIMENT

Hereinafter, the embodiments of the present invention will be described in detail with reference to the accompanying diagrams so that those skilled in the art can easily implement the invention. However, the description of the present invention is merely an example for structural and functional explanation, and the scope of the present invention should not be interpreted as being limited to the embodiments described in the text. That is, the embodiments can undergo various modifications and can take various forms, so the scope of the present invention should be understood to include equivalents that can realize the technical idea. Moreover, the purposes or effects presented in the present invention do not mean that a specific embodiment must include all of them or that only those effects should be included, so the scope of the present invention should not be understood as being limited thereby.

The meanings of terms described in the present invention should be understood as follows.

Terms like “first” and “second” are used to distinguish one component from another and do not limit the scope of the inventions. For example, the first component may be referred to as the second component, and similarly, the second component may be referred to as the first component. When a component is said to be “connected” to another component, it may be directly connected to that other component or there may be other components in between. In contrast, when a component is said to be “directly connected” to another component, it means there are no other components in between. Likewise, other expressions describing the relationship between components, such as “between” and “directly between” or “adjacent to” and “directly adjacent to,” should be interpreted similarly.

Singular expressions should be understood to include plural expressions unless the context clearly indicates otherwise. The terms “comprise” or “have” are intended to specify that there are features, numbers, steps, operations, components, parts, or combinations thereof, and do not preclude the possibility of the presence or addition of one or more other features, numbers, steps, operations, components, parts, or combinations thereof.

All terms used herein should be understood to have the same meaning as commonly understood by those skilled in the art unless otherwise defined. Terms defined in commonly used dictionaries should be interpreted as having meanings consistent with the context of the related technology and should not be interpreted as having idealized or excessively formal meanings unless explicitly defined in the present invention.

Configuration of the brain stimulation system

Hereinafter, the configuration of a preferred embodiment will be described in detail with reference to the accompanying diagrams.

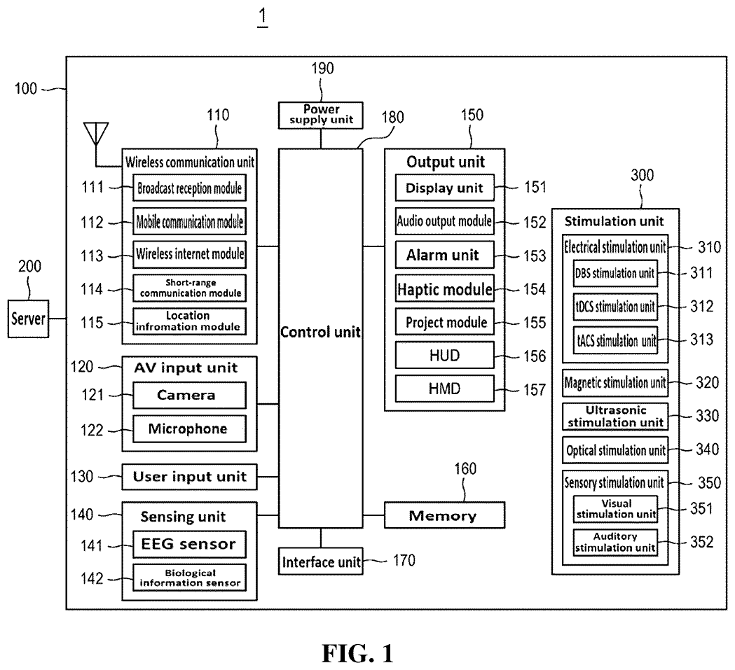

FIG. 1 is a block diagram of a brain stimulation system according to one embodiment of the present invention.

Referring to FIG. 1, the brain stimulation system (1) may comprise a brain stimulation device (100) and a server (200).

First, the brain stimulation device (100) may comprise a wireless communication unit (110), an A/V (Audio/Video) input unit (120), a user input unit (130), a sensing unit (140), an output unit (150), a memory (160), an interface unit (170), a control unit (180), a power supply unit (190), and a stimulation unit (300).

However, the components shown in FIG. 1 are not essential, and a brain stimulation system (1) with more or fewer components may be implemented.

Hereinafter, the components will be described in order.

The wireless communication unit (110) may comprise one or more modules that enable wireless communication between the brain stimulation system (1) and a wireless communication system or between devices located on a network.

For example, the wireless communication unit (110) may comprise a mobile communication module (112), a wireless internet module (113), a short-range communication module (114), and a location information module (115).

The broadcast reception module (111) receives broadcast signals and/or broadcast-related information from an external broadcast management server through a broadcast channel.

The broadcast channel may comprise satellite and terrestrial channels. The broadcast management server refers to a server that generates and transmits broadcast signals and/or broadcast-related information or receives and transmits pre-generated broadcast signals and/or broadcast-related information to the brain stimulation device (100). The broadcast signal may include TV broadcast signals, radio broadcast signals, data broadcast signals, as well as combined forms of TV or radio broadcast signals with data broadcast signals.

The broadcast-related information may refer to information related to broadcast channels, broadcast programs, or broadcast service providers.

The broadcast-related information may also be provided through a mobile communication network and received by the mobile communication module (112).

The broadcast-related information may exist in various forms, such as EPG (Electronic Program Guide) of DMB (Digital Multimedia Broadcasting) or ESG (Electronic Service Guide) of DVB-H (Digital Video Broadcast-Handheld).

The broadcast reception module (111) may receive digital broadcast signals using digital broadcast systems such as DMB-T (Digital Multimedia Broadcasting-Terrestrial), DMB-S (Digital Multimedia Broadcasting-Satellite), MediaFLO (Media Forward Link Only), DVB-H (Digital Video Broadcast-Handheld), DVB-CBMS, OMA-BCAST, ISDB-T (Integrated Services Digital Broadcast-Terrestrial), among others. Of course, the broadcast reception module (111) may be configured to be compatible with other broadcast systems besides the digital broadcast systems mentioned above.

Broadcast signals and/or broadcast-related information received through the broadcast reception module (111) may be stored in the memory (160).

The mobile communication module (112) transmits and receives wireless signals with a base station, an external brain stimulation device (100), or at least one server on a mobile communication network. The wireless signals may include various forms of data for voice calls, video calls, or text/multimedia message transmission and reception.

The wireless internet module (113) refers to a module for wireless internet access and may be built-in or external to the brain stimulation device (100).

Technologies for wireless internet may include WLAN (Wireless LAN) (Wi-Fi), Wibro (Wireless broadband), Wimax (World Interoperability for Microwave Access), HSDPA (High Speed Downlink Packet Access), etc.

The short-range communication module (114) refers to a module for short-range communication. Technologies for short-range communication may include Bluetooth, RFID (Radio Frequency Identification), infrared communication (IrDA, infrared Data Association), UWB (Ultra-Wideband), ZigBee, etc.

The location information module (115) is a module for obtaining the location of the brain stimulation device (100), and a representative example is a GPS (Global Positioning System) module. According to current technology, the GPS module (115) calculates distance information from three or more satellites and accurate time information, then applies triangulation to the calculated information to accurately calculate the current location information based on latitude, longitude, and altitude. Currently, a method of calculating location and time information using three satellites and correcting errors in the calculated location and time information using another satellite is widely used. Additionally, the GPS module (115) can calculate speed information by continuously calculating the current location in real-time.

Referring to FIG. 1, the A/V (Audio/Video) input unit (120) is for inputting audio or video signals and may comprise a camera (121) and a microphone (122).

The camera (121) processes image frames such as still images or videos obtained by an image sensor in shooting mode, and the processed image frames may be displayed on the display unit (151).

The image frames processed by the camera (121) may be stored in the memory (160) or transmitted externally through the wireless communication unit (110).

The camera (121) may be equipped with two or more cameras depending on the usage environment.

The microphone (122) inputs external sound signals through a microphone in recording mode or voice recognition mode and processes them into electrical voice data.

The processed voice data may be converted into a form transmittable to a mobile communication base station through the mobile communication module (112).

Various noise reduction algorithms may be implemented in the microphone (122) to remove noise generated during the process of inputting external sound signals.

Next, the user input unit (130) generates input data for controlling the operation of the brain stimulation system (1).

The user input unit (130) may be composed of a key pad, dome switch, touch pad (resistive/capacitive), jog wheel, jog switch, etc.

The sensing unit (140) detects the current state of the brain stimulation system (1) such as the open/closed state of the brain stimulation system (1), the position of the brain stimulation system (1), user contact, the orientation of the brain stimulation system (1), acceleration/deceleration of the brain stimulation system (1), etc., and generates sensing signals for controlling the operation of the brain stimulation system (1).

The sensing unit (140) may also sense whether the power supply unit (190) is supplying power, whether the interface unit (170) is connected to an external device, etc.

In particular, the sensing unit (140) according to the present invention may comprise an EGG sensor (141) and a biological information sensor (142) for measuring the biological information of an object (patient).

In the present invention, the biological information of an object is not limited but may include at least one of heart rate variability (HRV) information, electroencephalogram (EEG) information, heart rate information, stress information, body composition information, weight information, oxygen saturation information, pulse information, blood pressure information, iris information, voice information, vein information, and electrocardiogram (ECG) information.

Further, the EGG sensor (141) refers to a sensor for measuring the brain wave information of an object, and the biological information sensor (142) may be a sensor for measuring the remaining biological information excluding the brain wave information measurable by the EGG sensor (141).

That is, the sensing unit (140) is preferably understood as a sensor for measuring the aforementioned biological information.

Brain waves are generated by the communication between neurons in the brain, and these waves are synchronized electrical waves generated as neurons in the cerebral cortex transmit signals to each other. Brain waves can be measured through an electroencephalogram (EEG) test that measures the potential difference between surface electrodes placed on specific areas of the scalp. The brain waves shown in an electroencephalogram are the sum of the electrical activities of numerous cortical neurons under the surface electrodes.

Brain waves appear in various frequency bands, which indicate the state of the brain. Brain waves are classified into delta waves, theta waves, alpha waves, beta waves, and gamma waves according to their frequency bands.

Delta waves have a frequency band of less than 4 Hz and are characterized by large amplitudes. They appear in deep sleep states without dreams.

Theta waves have a frequency of 4-7 Hz and occur in certain sleep states and deep meditation. Theta waves are also known to be involved in the consolidation of memories during sleep.

Alpha waves have a frequency of approximately 8-13 Hz and appear in a state of relaxed wakefulness.

Beta waves have a frequency of 14-29 Hz and are the rhythm of an activated cerebral cortex. They appear during cognitive activities of the cerebral cortex in an awake state. Gamma waves have a frequency of 30-80 Hz and are high-frequency brain waves that appear in tense or excited states. They are known to appear in states of high concentration.

An electroencephalogram (EEG) is a non-invasive technique for measuring brain waves by fixing surface electrodes to the scalp with conductive gel and measuring the potential difference between the electrodes.

Several electrodes are attached to standard positions on the scalp, and the small amplitude voltage changes generated by cortical neurons are amplified by a signal amplifier and recorded by an brain wave recorder.

Brain waves are generated by synaptic currents of dendrites, which are currents that occur during the communication process of neurons in the cerebral cortex located just below the skull.

The synaptic current of a single neuron is very weak, and the signal must pass through multiple layers, including the meninges, cerebrospinal fluid, skull, and scalp, to reach the electrodes attached to the scalp. The reason that the electrical recording of brain waves is possible is that the brain waves are the sum of signals generated when thousands of neurons are activated together. Therefore, the more synchronized the activity of the neurons, the larger the amplitude and the lower the frequency of the brain waves.

Meanwhile, the output unit (150) generates outputs related to visual, auditory, or tactile senses, and may comprise a display unit (151), a sound output module (152), an alarm unit (153), a haptic module (154), a projector module (155), a head-up display (HUD), and a head-mounted display (HMD).

The display unit (151) displays (outputs) information processed in the brain stimulation system (1).

The display unit (151) may comprise at least one of a liquid crystal display (LCD), a thin-film transistor-liquid crystal display (TFT-LCD), an organic light-emitting diode (OLED), a flexible display, and a 3D display.

Some of these displays may be configured to be transparent or see-through so that the outside can be seen through them. This is called a transparent display, with a representative example being a TOLED (Transparent OLED). The rear structure of the display unit (151) may also be configured to be light-transmitting. Through this structure, the user can see objects located behind the body of the brain stimulation system (1) through the area occupied by the display unit (151) of the body of the brain stimulation system (1).

Depending on the implementation form of the brain stimulation system (1), there may be two or more display units (151). For example, the brain stimulation system (1) may have multiple display units arranged spaced apart on one surface or integrated, and they may also be arranged on different surfaces, respectively.

When the display unit (151) and a sensor that detects touch operations (hereinafter referred to as a “touch sensor”) form a mutual layer structure (hereinafter referred to as a “touch screen”), the display unit (151) may be used not only as an output device but also as an input device. The touch sensor may be in the form of, for example, a touch film, a touch sheet, or a touch pad.

The touch sensor may be configured to convert changes such as pressure applied to a specific part of the display unit (151) or changes in capacitance occurring at a specific part of the display unit (151) into electrical input signals. The touch sensor may be configured to detect not only the position and area of the touch but also the pressure during the touch.

When there is a touch input to the touch sensor, the corresponding signal(s) are sent to the touch controller. The touch controller processes the signal(s) and then sends the corresponding data to the control unit (180). Thus, the control unit (180) can determine which area of the display unit (151) has been touched.

The proximity sensor may be placed inside the brain stimulation system (1) enclosed by the touch screen or near the touch screen. The proximity sensor detects the presence or absence of an object approaching a predetermined detection surface or existing nearby using an electromagnetic field or infrared without mechanical contact. Proximity sensors have a longer lifespan than contact sensors and are highly versatile.

Examples of proximity sensors include transmissive photoelectric sensors, direct reflection photoelectric sensors, mirror reflection photoelectric sensors, high-frequency oscillation proximity sensors, capacitive proximity sensors, magnetic proximity sensors, and infrared proximity sensors. If the touch screen is capacitive, it detects the approach of the pointer by the change in the electric field caused by the proximity of the pointer. In this case, the touch screen (touch sensor) may be classified as a proximity sensor.

The proximity sensor detects a proximity touch and a proximity touch pattern (e.g., proximity touch distance, proximity touch direction, proximity touch speed, proximity touch time, proximity touch location, proximity touch movement status, etc.). Information corresponding to the detected proximity touch action and proximity touch pattern may be output on the touch screen.

The sound output module (152) can output audio data received from the wireless communication unit (110) or stored in the memory (160) in recording mode, voice recognition mode, broadcast reception mode, etc.

The sound output module (152) also outputs sound signals related to the functions performed by the brain stimulation system (1). This sound output module (152) may comprise a receiver, speaker, and buzzer.

The alarm unit (153) outputs signals to notify the occurrence of events in the brain stimulation system (1).

The alarm unit (153) may output signals to notify the occurrence of events in forms other than video or audio signals, for example, vibration.

The video or audio signals can also be output through the display unit (151) or the sound output module (152), so they (151, 152) may be classified as part of the alarm unit (153).

The haptic module (154) generates various tactile effects that the user can feel. A representative example of the tactile effects generated by the haptic module (154) is vibration. The intensity and pattern of the vibrations generated by the haptic module (154) are controllable.

For example, different vibrations may be synthesized and output, or output sequentially.

The haptic module (154) can generate various tactile effects in addition to vibration, such as pin arrays that move vertically relative to the contact skin surface, air jet forces or suction forces through nozzles or suction ports, brushing against the skin surface, stimulation by contact with electrodes, electrostatic forces, and the reproduction of cold and heat sensations using elements that can absorb or generate heat.

The haptic module (154) may be implemented to deliver tactile effects not only through direct contact but also so that the user can feel the tactile effects through muscle senses in their fingers or arms. Depending on the implementation form of the brain stimulation system (1), there may be two or more haptic modules (154).

The projector module (155) is a component for performing an image projection function using the brain stimulation system (1) and can display the same or at least partly different images as those displayed on the display unit (151) on an external screen or wall, according to the control signals of the control unit (180).

Specifically, the projector module (155) may comprise a light source (not shown) for generating light (e.g., laser light) to output images externally, an image generation means (not shown) for generating images to be output externally using the light generated by the light source, and a lens (not shown) for enlarging and outputting the images at a predetermined focal distance. Additionally, the projector module (155) may comprise a device (not shown) for mechanically moving the lens or the entire module to adjust the direction of image projection.

The projector module (155) may be classified into CRT (Cathode Ray Tube) modules, LCD (Liquid Crystal Display) modules, and DLP (Digital Light Processing) modules depending on the type of display device element. In particular, the DLP module may be advantageous for miniaturizing the projector module (151) by generating images through the reflection of light generated by the light source on a DMD (Digital Micromirror Device) chip.

Preferably, the projector module (155) may be provided on the side, front, or rear of the brain stimulation system (1) in the longitudinal direction. Of course, the projector module (155) may be provided in any position of the brain stimulation system (1) as needed.

The head-up display (HUD, 156) is a device that projects graphic images of current vehicle speed, remaining fuel, navigation directions, etc., onto the windshield directly in front of the driver in vehicles.

The head-mounted display (HMD, 157) is a representative device capable of outputting virtual reality (VR) information.

Virtual reality (VR) refers to a human-computer interface technology that creates a specific environment or situation as three-dimensional (3D) content using a computer, making the user feel as if they are interacting with the actual surrounding environment.

Generally, the stereoscopic perception that people experience is created by a complex combination of various factors, such as the degree of change in lens thickness according to the position of the observed object, the angle difference between the two eyes and the object, the difference in the position and shape of the object seen by each eye, the parallax caused by the movement of the object, and various psychological and memory effects.

The most important factor in stereoscopic perception is binocular disparity, which occurs because the two eyes are approximately 65 cm apart horizontally. That is, due to binocular disparity, the angle difference when viewing an object causes different images to enter each eye, and when these two images are transmitted to the brain through the retinas, the brain precisely fuses these two pieces of information to perceive the original 3D stereoscopic image.

These stereoscopic 3D contents have already been widely used and well-received by consumers in various media fields, such as 3D movies, 3D games, and experience displays.

With the popularization of 3D contents using virtual reality technology, there is an increasing demand for the development of technologies that can provide more immersive virtual reality services.

Generally, an image display device refers to an image display device that forms a focus so that an enlarged virtual screen may be composed at a long distance by utilizing precise optical devices with image light generated at a very close distance to the eyes.

Image display devices may be classified into see-close type, where only the image light emitted from the display device can be seen and the surrounding environment cannot be seen, and see-through type, where both the surrounding environment through a window and the image light emitted from the display device can be seen simultaneously.

The head-mounted display (HMD, 157) according to the present invention refers to various digital devices that are worn on the head like glasses to provide multimedia content. In line with the trend toward lightweight and miniaturization of digital devices, various wearable computers are being developed, and HMDs are also widely used.

The HMD (157) can provide various conveniences to users by combining with technologies such as augmented reality and N-screen technology, beyond the simple display function.

For example, when a microphone and a speaker are attached to the HMD (157), the user can make phone calls while wearing the HMD (157). Additionally, when a far-infrared camera (122) is attached to the HMD (157), the user can capture images in the desired direction while wearing the HMD (157).

The memory unit (160) can store programs for processing and control by the control unit (180), and can also temporarily store data being input/output (e.g., messages, audio, still images, videos, etc.). The memory unit (160) can also store the usage frequency of each of the data. Additionally, the memory unit (160) can store data on various patterns of vibrations and sounds output when there is touch input on the touch screen.

The memory (160) may comprise at least one type of storage medium such as a flash memory type, hard disk type, multimedia card micro type, memory card (e.g., SD or XD memory), RAM (Random Access Memory), SRAM (Static Random Access Memory), ROM (Read-Only Memory), EEPROM (Electrically Erasable Programmable Read-Only Memory), PROM (Programmable Read-Only Memory), magnetic memory, magnetic disk, and optical disk. The brain stimulation system (1) can operate in connection with web storage performing the storage function of the memory (160) on the internet.

The interface unit (170) serves as a passage for all external devices connected to the brain stimulation system (1). The interface unit (170) transmits data received from an external device or power supplied from an external device to each component inside the brain stimulation system (1), or transmits data from inside the brain stimulation system (1) to an external device. For example, the interface unit (170) may comprise a wired/wireless headset port, an external charger port, a wired/wireless data port, a memory card port, a port for connecting a device equipped with an identification module, an audio I/O port, a video I/O port, an earphone port, etc.

The identification module is a chip storing various information for authenticating the usage rights of the brain stimulation system (1), and may comprise a user identify module (UIM), a subscriber identify module (SIM), a universal subscriber identity module (USIM), etc. A device equipped with an identification module (hereinafter referred to as an “identification device”) may be manufactured in the form of a smart card. Therefore, the identification device may be connected to the brain stimulation system (1) through a port.

The interface unit comprises a passage through which power from the cradle is supplied to the brain stimulation system (1) when the brain stimulation system (1) is connected to an external cradle, or a passage through which various command signals inputted by the user from the cradle are transmitted to the brain stimulation device. Various command signals or power input from the cradle may operate as signals for recognizing that the mobile device is correctly mounted on the cradle.

The control unit (180) typically controls the overall operation of the brain stimulation system (1).

In the present invention, the control unit (180) may be provided in the brain stimulation device (100), but is not limited to this, and preferably, it may be implemented as an app (application) pre-stored (or installed) on a device (e.g., smartphone, tablet, PC, etc.) owned by the user of the brain stimulation device (100).

The power supply unit (190) supplies the power required for the operation of each component by receiving external or internal power under the control of the control unit (180).

The various embodiments described herein may be implemented, for example, using software, hardware, or a combination thereof within a computer or similar device readable recording medium.

In terms of hardware implementation, the embodiments described herein may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and other electrical units for performing specific functions. In some cases, the embodiments described herein may be implemented as the control unit (180) itself.

In terms of software implementation, the procedures and functions described in the present specification may be implemented as separate software modules. Each of the software modules can perform at least one of the functions and operations described in the present specification. The software code may be implemented as a software application written in a suitable programming language. The software code may be stored in the memory (160) and executed by the control unit (180).

Additionally, the stimulation unit (300) provides a function to stimulate the user's brain through neurostimulation techniques, neurofeedback techniques, sensory stimulation techniques, etc.

The stimulation unit (300) according to the present invention may be provided in the brain stimulation device (100) and may comprise an electrical stimulation unit (310), a magnetic stimulation unit (320), an ultrasound stimulation unit (330), an optical stimulation unit (340), and a sensory stimulation unit (350).

First, the electrical stimulation unit (310) may comprise a DBS stimulation unit (311), a tDCS stimulation unit (312), and a tACS stimulation unit (313).

The DBS stimulation unit (311) uses deep brain stimulation (DBS), which involves placing fine electrodes in the deep nuclei of the brain to stimulate the activity of neurons. DBS stimulation may be provided in the form of DC, pulse, or NIR.

Regarding deep brain stimulation (DBS), applying electrical stimulation to the nuclei of specific brain regions can interfere with pathological signals occurring in brain areas, thereby aiming to treat and improve symptoms of various diseases, including motor disorders.

Deep brain stimulation involves placing fine electrodes in the deep nuclei of the brain and receiving the necessary power for activity from a pulse generator implanted in the chest, similar to a pacemaker.

Through this, depolarization block, i.e., blocking the neural output of neurons located at the electrode site, may be achieved.

Additionally, synaptic inhibition, i.e., indirectly regulating the output of neurons by activating the axon terminals that have synaptic connections to the neurons near the electrodes, may be achieved.

Next, the tDCS stimulation unit (312) uses transcranial direct current stimulation (tDCS), which involves attaching electrodes to the head and stimulating the neurons in the cerebral cortex with a weak direct current.

The tDCS stimulation unit (312) is a non-invasive brain stimulation method for recovering sequelae caused by brain damage and can help improve brain function by regulating the activation state of brain neurons through electrical stimulation.

The tACS (transcranial Alternating Current Stimulation) stimulation unit (313) involves attaching electrodes and delivering microcurrents of less than 1 mA to the skull, and is used in non-pharmacological treatment to improve symptoms of anxiety, depression, insomnia, stress, headaches, and various types of pain.

tACS (transcranial Alternating Current Stimulation) stimulation is effective in regulating microglia, is safe with microcurrent usage, and has no side effects, allowing for long-term treatment.

Further, it is an advanced therapy highly compatible with existing chemical therapies that promote and/or inhibit hormone secretion.

When applying tACS (transcranial Alternating Current Stimulation) stimulation, it is possible to induce sleep and improve sleep quality by maintaining a stable DMN of the brain itself.

Moreover, it is possible to induce sleep and improve sleep quality through the improvement of hormones (serotonin, melatonin, GABA, etc.).

Further, it can stimulate brain tissue to restore neurochemicals to their pre-stress balance.

The magnetic stimulation unit (320) uses TMS (transcranial magnetic stimulation), also known as transcranial magnetic stimulation, and non-invasively stimulates neurons in the brain using magnetic energy.

It is effective in treating neurological and psychiatric disorders such as Parkinson's syndrome and depression.

When a strong magnetic field is generated near the head with a conducting electromagnetic coil, this magnetic field passes through the skull and stimulates the neurons in the transcranial cortex.

At this time, the activity of the cerebral cortex can be increased or decreased depending on the speed of the magnetic field. For example, in cases where the activity of the cerebral cortex is low, such as depression, high-frequency stimulation is used, and in cases where the activity is too high, such as anxiety or mania, low-frequency stimulation is used to regulate the activity.

In addition, the ultrasound stimulation unit (330) utilizes ultrasound for therapeutic effects and is widely used in obstetrics and gynecology, orthopedics, dermatology, and more.

Ultrasound refers to sound waves with frequencies beyond the hearing limit of humans. Generally, sound waves that a healthy person can hear are up to 20 kHz, and ultrasound refers to sound waves beyond this frequency. Ultrasound therapy utilizes such ultrasound for therapeutic effects and is widely used in obstetrics and gynecology, orthopedics, dermatology, and more.

Ultrasound therapy can be broadly divided into high-intensity ultrasound of 1000 W/cm² or more and low-intensity ultrasound in the range of 10-50 W/cm². High-intensity ultrasound therapy selectively heats tissues for treatment and is mainly used for tumor treatment, while low-intensity ultrasound therapy heats subcutaneous tissues for treatment and is used in musculoskeletal treatments such as skin lifting, fracture, and cartilage cell regeneration. Moreover, ultrasound therapy has the advantage of quick recovery as it does not damage the skin.

HIFU (High Intensity Focused Ultrasound) uses heat and energy generated by focusing ultrasound beams emitted from multiple directions to necrotize or reduce the size of tumors without incision or surgery. It is used in treating uterine fibroids, prostate cancer, bone metastasis cancer, liver cancer, and more.

LIFU (Low Intensity Focused Ultrasound) is a method that achieves skin lifting effects through subcutaneous tissue necrosis using heat. It is similar to high-intensity focused ultrasound in therapeutic mechanism but differs in intensity and range used.

LIPUS (Low Intensity Pulsed Ultrasound) stimulates physical vibration by radiating ultrasound to the treatment area to activate cells, and is used in treatments such as fracture healing and cartilage cell regeneration.

Sonophoresis uses low-frequency ultrasound to deliver drugs through the skin.

Further, the optical stimulation unit (340) applies the Brain Photo Modulation method by irradiating light on the head to stimulate the brain.

According to the optical stimulation unit (340), light of 600-1000 nm penetrates the cell wall and engages in the mitochondrial COX (Cytochrome c oxidase) respiratory chain.

Through this, it is possible to increase synapse creation, increase vascular formation, increase blood flow, prevent inflammation, prevent cell apoptosis, increase SOD, and decrease neurotoxicity.

Further, the sensory stimulation unit (350) may comprise a visual stimulation unit (351) and an auditory stimulation unit (352).

The sensory stimulation unit (350) stimulates other organs indirectly to stimulate the brain without directly stimulating the brain.

The visual stimulation unit (351) refers to radiation that can cause a sensation of light to the naked eye.

For example, the visual stimulation unit (351) uses the flash of a white stroboscope of about 100,000 lux, fixes the stroboscope valve about 20 cm in front of the closed eyes of the subject, and illuminates the entire visual field. The stimulation method gives stimulation for 10 seconds in the order from low frequency to high frequency, then observes the brain waves or the subject's condition for 10 seconds before moving to the next stimulation.

Meanwhile, the server (200) can exchange information with the brain stimulation device (100) by establishing a database.

At this time, short-distance communication or long-distance communication may be applied between the server (200) and the brain stimulation device (100).

Further, the server (200) may form a network with medical institutions to receive judgment and opinion-related information from medical staff and deliver it to the brain stimulation device (100).

### Structure of the Brain Stimulation Device

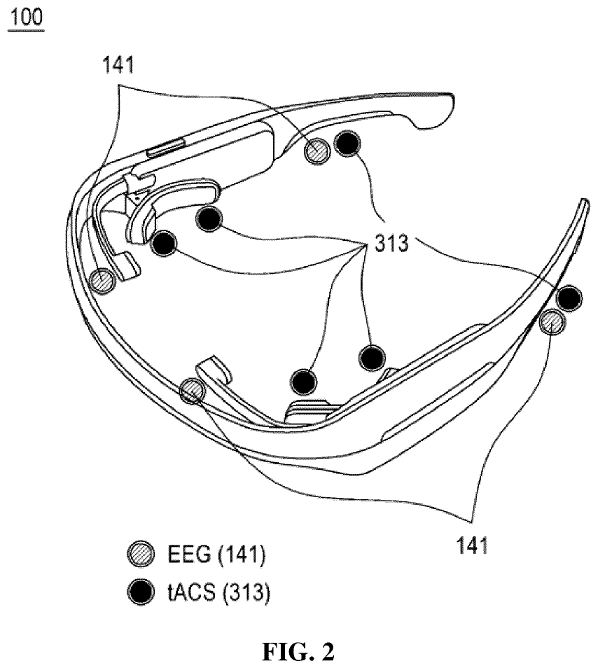

FIG. 2 is a diagram illustrating an example of a brain stimulation device.

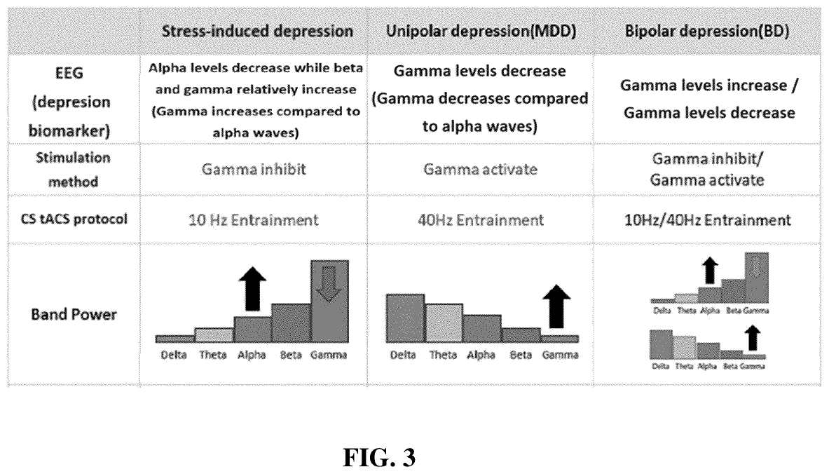

Referring to FIG. 2, the brain stimulation device (100) may be manufactured in a form that may be worn on the head to stimulate the brain of the object (patient) as shown in FIG. 3.

At this time, the brain stimulation device (100) may have multiple sensing units (140) arranged to measure the biological information of the object.

In one embodiment, the brain stimulation device (100) may have multiple sensing units (140) implemented as EGG sensors (141) to measure the EEG information of the object, which is one of the biological information of the object.

Further, the brain wave information measured by the EGG sensor (141) may be the first brain wave obtained from the brain of the object before the stimulation unit (300) delivers stimulation to the brain of the object. The EGG sensor (141) can measure not only the first brain wave but also the second brain wave of the object in the state where the stimulation unit (300) delivers stimulation to the brain of the object.

The sensing unit (140) may be implemented as a biological information sensor (142) that measures the biological information of the object excluding the brain wave (EGG) information, but in one embodiment, it will be described based on the EGG sensor (141).

Further, the brain stimulation device (100) may have multiple electrical stimulation units (310) arranged to deliver stimulation to the brain of the object for improving the depression of the object.

Multiple electrical stimulation units (310) may be tACS stimulation units (313) to deliver tACS-based complex stimulation to the brain of the object.

Further, although not shown in the diagram, the multiple electrical stimulation units (310) may comprise DBS stimulation units (311) and tDCS stimulation units (312), or be replaced by at least one of the DBS stimulation units (311) and tDCS stimulation units (312).

The method of delivering stimulation to the brain of the object through such a brain stimulation device (100) is as follows.

### Stimulation Method Related to the Present Invention

First, the sensing unit (140) can measure the biological information of the object (patient).

The control unit (180) can determine that the state of the object corresponds to a first state among the plurality of predetermined depressive states shown in FIG. 3 based on the biological information of the object measured by the sensing unit (140).

FIG. 3 is a diagram for explaining a plurality of predetermined depressive states related to depression.

In the present invention, the plurality of predetermined depressive states related to depression may include a depressive disorder state caused by stress (stress-induced depression), a major depressive disorder (MDD) state, and a bipolar disorder (BD) state, as shown in FIG. 3.

Meanwhile, the electrical stimulation unit (310) can deliver stimulation that varies according to the first state of the object determined by the control unit (180) to entrain synchronized oscillations in multiple regions of the brain of the object.

In the present invention, the stimulation delivered to the brain of the object may be the first stimulation to entrain gamma oscillation synchronized in multiple regions of the brain, the second stimulation to entrain any one of delta, theta, alpha, and beta oscillations synchronized in multiple regions of the brain, or the third stimulation combining the first stimulation and the second stimulation.

The first stimulation, the second stimulation, and the third stimulation may each be transcranial alternating current stimulation (tACS, 400) delivered to the brain of the object by the electrical stimulation unit (310).

That is, it is preferable that the electrical stimulation unit (310) is configured as a tACS stimulation unit (313) capable of delivering tACS stimulation (400) to the brain of the object.

The tACS stimulation unit (313) can deliver the first stimulation to the brain of the object when the first state of the object determined by the control unit (180) is a major depressive disorder state.

At this time, the delivery of the first stimulation is to enhance gamma-level brain waves in the brain waves of the object through the entrainment of gamma oscillations as shown in FIG. 3.

The tACS stimulation unit (313) can deliver the second stimulation to the brain of the object when the first state of the object determined by the control unit (180) is a depressive disorder state caused by stress.

At this time, the delivery of the second stimulation is to reduce gamma-level brain waves in the brain waves of the object through the entrainment of any one of delta, theta, alpha, and beta oscillations as shown in FIG. 3.

The tACS stimulation unit (313) can deliver the third stimulation to the brain of the object when the first state of the object determined by the control unit (180) is a bipolar disorder state.

More specifically, when the first state of the object is a bipolar disorder state, and the gamma-level brain waves are at their lowest in the brain waves of the object, the tACS stimulation unit (313) can deliver the first stimulation to the brain of the object to enhance the gamma-level brain waves.

Conversely, when the first state of the object is a bipolar disorder state, and the gamma-level brain waves are at their highest in the brain waves of the object, the tACS stimulation unit (313) can deliver the second stimulation to the brain of the object to reduce the gamma-level brain waves.

tACS Stimulation Method Proposed by the Present Invention

It was previously impossible to induce the entrainment of synchronized gamma oscillations with tACS stimulation as proposed by the present invention and to sense this in real-time.

That is, for the Gamma Entrainment Therapy (GET) method, real-time monitoring of brain signals is essential. However, since gamma oscillations range from about 25-100 Hz, usually targeting 40 Hz, and because the stimulation is applied at 40 Hz, real-time monitoring is technically impossible due to the same frequency range of the stimulation frequency and the EEG measurement frequency.

For example, when giving a 40 Hz stimulation to entrain gamma oscillations, the sensing through EEG also targets the 40 Hz signal band, making it difficult to distinguish whether the measured signal is noise from the stimulation or the target EEG signal, which was a significant problem preventing the implementation of the method proposed by the present invention.

In contrast, the present invention can separate the frequency bands of EEG and stimulation frequency using a composite stimulation method with Burst Frequency (Burst) and Pulse Repetition Frequency (PRF) for real-time EEG signal acquisition.

Moreover, the frequency of the composite stimulation follows the Pulse Repetition Frequency (PRF) in the 30 Hz to 50 Hz range to induce the entrainment of synchronized oscillations in multiple brain regions of the object.

The composite stimulation has a signal magnitude to induce membrane action potential and brain oscillation in multiple brain regions of the object, and this signal is not continuously applied but has the form of a burst signal (Burst Signal) that is turned on (ON) and off (OFF) according to the PRF.

Further, the signal turned on (ON) according to the PRF is applied as stimulation according to the Burst Frequency, and at this time, the Burst Frequency (Burst) is set higher than the Pulse Repetition Frequency (PRF).

For example, the PRF that is turned on (ON) and off (OFF) may use a low frequency of 40 Hz (for the purpose of inducing gamma oscillation entrainment), and the Burst frequency of the signal turned on (ON) may use a high frequency of 10 kHz (for the purpose of inducing membrane action potential and brain oscillation).

The signal caused by the composite stimulation is in the high-frequency (10 kHz) band, so to measure the target EEG signal frequency (40 Hz), it is separated through a low-pass filter. Also, since the magnitude of the electrical signal generated by the PRF is about 100 times smaller than the magnitude of the target EEG signal, it is possible to acquire the brain wave signal induced by the PRF of the composite stimulation in real-time without interference.

That is, if electrical stimulation is given at 40 Hz to receive the 40 Hz EEG in real-time, real-time monitoring is not possible due to interference between the electrical stimulation signal and the EEG signal.

Therefore, in the present invention, gamma entrainment is induced by delivering high-frequency burst electrical stimulation through a 40 Hz PRF.

At this time, since the electrical stimulation signal due to the burst is distinguished as a high-frequency band and may be separated, it is possible to sense the desired 40 Hz EEG signal in real-time.

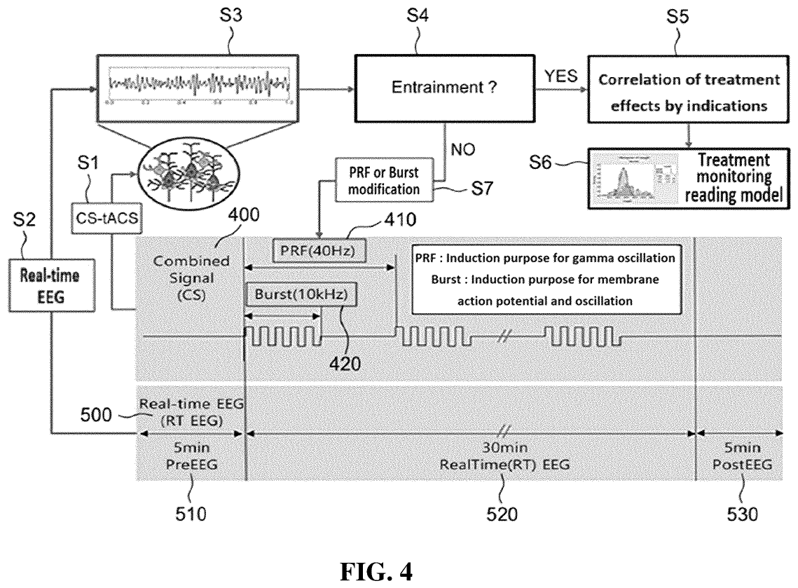

FIG. 4 will be referred to for a more detailed explanation.

FIG. 4 is a diagram for explaining the EEG signal-based closed-loop neurofeedback tACS stimulation method proposed by the present invention.

Referring to FIG. 4, the brain stimulation device (100) can utilize the tACS stimulation (400) method in a composite stimulation method with Burst Frequency (Burst) and Pulse Repetition Frequency (PRF) to separate the frequency bands of EEG and stimulation frequency.

At this time, the composite stimulation tACS (400) is a combined signal that repeats on (ON) and off (OFF) according to the predetermined first frequency (410), which is the Pulse Repetition Frequency (PRF), and the signal turned on (ON) according to the first frequency (410) is applied as stimulation according to the predetermined second frequency (420), which is the Burst Frequency (Burst).

That is, the tACS stimulation (400) may be the first combined stimulation signal comprising the first frequency (410) and the second frequency (420).

As described above, the PRF (410) is applied to induce the entrainment of synchronized oscillations in multiple brain regions of the object, and the Burst Frequency (420) is applied to induce membrane action potential and brain oscillation in multiple brain regions of the object.

The Burst Frequency (420) is set to 10 kHz, which is a higher value than the Pulse Repetition Frequency (410) of 40 Hz.

The Burst Frequency (420) moves neurons through membrane action potential, inducing brain oscillation and enabling communication between neurons.

By stimulating with a high-frequency Burst Frequency (420), it is possible to easily induce membrane action and quickly induce oscillation. Furthermore, to distinguish between the brain wave signal induced by the PRF (410) of the composite stimulation generated in the brain and the applied composite stimulation, the Burst Frequency (420) is set to a much higher frequency than the 30 Hz to 50 Hz band.

In addition, the Pulse Repetition Frequency (410) is applied to induce the entrainment of synchronized gamma oscillations, and the activity stimulated by the Burst Frequency (420) is induced to be synchronized and entrained according to the gamma oscillation.

In particular, the Pulse Repetition Frequency (410) can deliver stimulation with a frequency of 30 Hz to 50 Hz to the object to entrain synchronized gamma oscillations in multiple brain regions, including the prefrontal cortex (PFC) and hippocampus of the object.

When composite stimulation is applied to the brain of the object, neuron activity is induced by the Burst Frequency (420), causing irregular communication, and the neurons activated in the gamma wave region by the PRF (410) are entrained to operate.

Therefore, the present invention induces brain wave entrainment through high-frequency burst electrical stimulation and gamma entrainment through a 40 Hz PRF.

Further, since the Burst Frequency (420) of the applied composite stimulation is set to a much higher frequency than the 30 Hz to 50 Hz band, it is possible to distinguish between the brain wave signal induced by the PRF (410) of the composite stimulation and the applied composite stimulation, and to acquire only the actual target brain wave by ignoring the sensed composite stimulation.

Previously, it was impossible to measure in real-time because PRF was applied at 40 Hz and sensing had to be done at 40 Hz through EEG, but as in the present invention, when a burst signal that is turned on (ON) and off (OFF) according to the Burst Frequency (420) is applied as a composite stimulation according to the PRF (410) to induce the entrainment of synchronized oscillations in the brain regions, a difference of more than 40 dB (mathematically calculated as more than 100 times) is created, and it becomes possible to distinguish between the signal due to the composite stimulation and the brain wave signal induced by the PRF (410) of the composite stimulation and to acquire only the target brain wave signal.

The EGG sensor (141) can measure the brain waves induced by stimulation, and the control unit (180) determines whether a response is derived by using whether the brain waves measured by the EGG sensor (141) correspond to the output and waveform of the entrained brain waves that appear only when gamma oscillations are entrained.

The control unit (180) extracts the properties of the measured brain waves by calculating the average and standard deviation of the power spectrum values of each frequency band from the brain waves of the object measured by the EGG sensor (141), and the ratios of the average values according to the combinations of gamma (gamma), alpha (alpha), beta (beta), delta (delta), and theta (theta) brain waves.

The EEG sensor (141) according to the present invention measures the EGG signal (500) in real-time and can sense the pre-EEG signal (510), which is not used for the first 5 minutes, the RT EEG signal (520), which is used for real-time analysis for the next 20 minutes, and the post-EEG signal (530), which is treated as an unused signal for another 5 minutes after measuring the RT EEG signal (520) for 20 minutes.

As described above, the purpose of measuring EEG in real-time in the present invention is to confirm whether the synchronized gamma oscillations are actually entrained in the brain regions through the device provided by the present invention.

Generally, synchronized gamma oscillations can be entrained by applying composite stimulation according to the 40 Hz PRF (410).

However, the entrainment of synchronized gamma oscillations may occur at a lower frequency than 40 Hz depending on the depressive state of the patient.

Therefore, in the present invention, the first frequency (410), the second frequency (420), and the output, waveform, and period of the stimulation according to the second frequency (420) are modified to deliver the second composite stimulation to the brain of the object to induce the actual entrainment of synchronized gamma oscillations.

As a specific example, if the PRF (410) is initially applied at 40 Hz and gamma oscillation entrainment is not induced, the entrainment can be induced by lowering the signal to 38, 36, 34, and 32 Hz, and through the continuous use of the brain stimulation device (100), the brain function of the patient can be improved so that entrainment is induced in the normal range of 40 Hz for patients initially entrained in the 32 Hz range.

Conversely, there may be patients in whom gamma oscillation entrainment is induced at a frequency higher than 40 Hz, so it is also possible to stimulate by modifying the composite stimulation in the direction of increasing the frequency.

Referring to FIG. 4, the first combined stimulation signal can be applied according to the PRF (410) to induce the entrainment of synchronized oscillations in the brain regions through the tACS stimulation unit (313), which is turned on (ON) and off (OFF) according to the Burst Frequency (420) (S1).

In addition, the EGG sensor (141) can measure the brain wave signal according to the first combined stimulation while the stimulation is being delivered to the brain of the object (S2, S3).

At this time, the control unit (180) can distinguish and ignore the first signal due to the first combined stimulation among the signals measured (sensed) by the EGG sensor (141) as noise.

Moreover, the control unit (180) can determine whether a response inducing gamma oscillation entrainment is derived based on the second signal, which is the brain wave signal induced by the PRF (410) of the composite stimulation, among the signals measured by the EGG sensor (141), excluding the first signal (S4).

If the synchronized gamma oscillation is actually entrained, and the first response according to the first combined stimulation is derived (S4-YES), the control unit (180) analyzes the correlation between the improvement (treatment) effect of depression according to the depressive condition (indication) of the patient and the induced response (S5) and provides the analysis result of the correlation in a table or graph form to the user through the display unit (151) (S6).

Conversely, if the synchronized gamma oscillation is not actually entrained, and the first response according to the first combined stimulation is not derived (S4-NO), the control unit (180) can control the tACS stimulation unit (313) to deliver the second composite stimulation, which at least one of the first frequency (410), the second frequency (420), the output, waveform, and period of the stimulation according to the second frequency (420) are modified, to the brain of the object (S7).

At this time, the first response refers to the entrainment of gamma oscillation or any one of delta, theta, alpha and beta oscillations synchronized in multiple brain regions.

Moreover, the first frequency (410) may be a frequency of 30 Hz to 80 Hz to entrain synchronized gamma oscillations in multiple brain regions, including the prefrontal cortex (PFC) and hippocampus of the object, a frequency of 14 Hz to 29 Hz to entrain beta oscillations, a frequency of 8 Hz to 13 Hz to entrain alpha oscillations, a frequency of 4 Hz to 7 Hz to entrain theta oscillations, or a frequency of more than0 Hz to less than 4 Hz to entrain delta oscillations.

Meanwhile, based on the magnitude of the first signal being different from the magnitude of the second signal by at least a predetermined value, the signal interference due to the first combined stimulation can be ignored.

Ultimately, the present invention can improve the depressive state of the patient through a neurofeedback method that determines whether gamma oscillation is actually achieved based on the EEG signal acquisition technology during real-time tACS stimulation.

Real-Time tACS-EEG Neurofeedback Algorithm

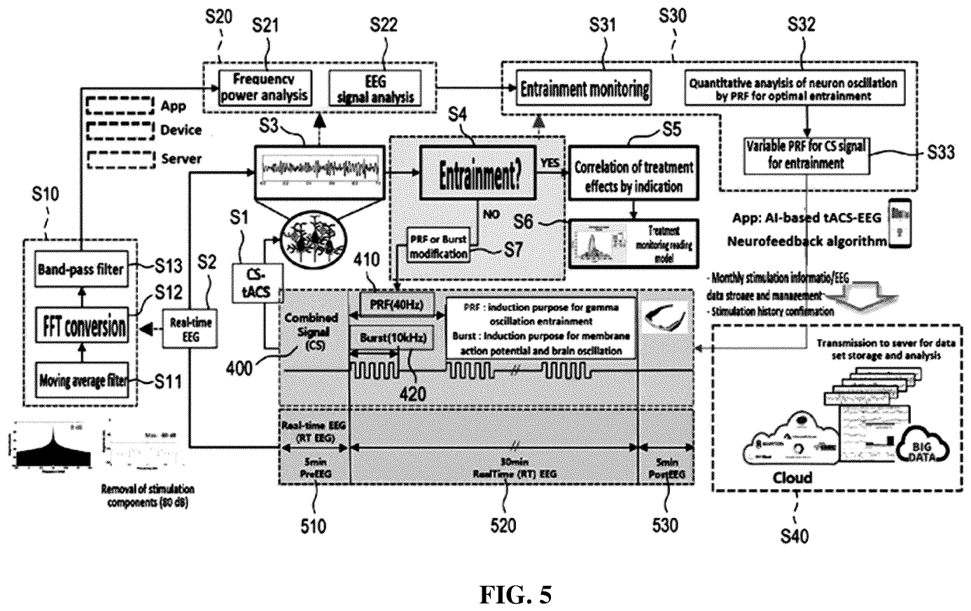

FIG. 5 is a block diagram of a real-time tACS-EEG Neurofeedback algorithm according to an embodiment of the present invention.

First, a real-time tACS-EEG Neurofeedback algorithm can be developed to induce optimized personalized gamma entrainment for each patient.

The real-time tACS-EEG Neurofeedback algorithm may be loaded onto the control unit (180) after undergoing a learning and validation process.

In addition, the control unit (180) can preprocess the brain wave signal according to the first combined stimulation from the EGG sensor (141) (S10).

In the preprocessing step (S10), when the control unit (180) receives the second signal from the brain wave signal according to the first combined stimulation from the EGG sensor (141), it may sequentially apply a moving average filter on the time axis (S11), then perform frequency axis conversion through FFT (S12), and apply a band-pass filter to remove the rated voltage and observe only the gamma (Gamma) band (S13).

Further, the control unit (180) can analyze the preprocessed second signal after the preprocessing step (S10) (S20).

In the analysis step (S20), the control unit (180) can perform power analysis of the frequency (S21) and analysis of the EGG signal (S22) simultaneously or sequentially to determine whether the first response is derived from the preprocessed second signal.

Moreover, after the analysis step (S20), the control unit (180) can induce optimized personalized gamma entrainment for each patient (S30).

In the gamma entrainment step (S30), the control unit (180) monitors the gamma entrainment (S31), quantitatively analyzes neuron oscillation by PRF (410) for optimized gamma entrainment for the patient (S32), and can vary the PRF (410) of the first combined stimulation for gamma entrainment according to the quantitative analysis results (S33).

Meanwhile, the control unit (180) ensures that the monthly stimulation information/EGG data of the patient using the brain stimulation device (100) is stored in the memory (160) and can transmit the monthly stimulation information/EGG data to the server (200) via the wireless communication unit (110) (S40).

Accordingly, the brain stimulation device (100) can receive the monthly stimulation information/EGG data from the server (200) via the wireless communication unit (110) when the user requests to check the monthly stimulation information/EGG data by touching the user input unit (130) or the display unit (150) and can provide the monthly stimulation information/EGG data to the user through the display unit (151).

Further, the control unit (180) ensures that the information of the first combined stimulation or the second combined stimulation delivered to the brain of the object (patient) included in the monthly stimulation information is stored in the memory (160) and can transmit the information of the first combined stimulation or the second combined stimulation to the server (200) via the wireless communication unit (110).

Accordingly, the brain stimulation device (100) can receive the information of the first combined stimulation or the second combined stimulation delivered to the brain of the object (patient) from the server (200) via the wireless communication unit (110) when the user requests to check the stimulation history delivered to the brain of the object by touching the user input unit (130) or the display unit (150) and can provide the stimulation history information to the user through the display unit (151).

### Clinical Trials

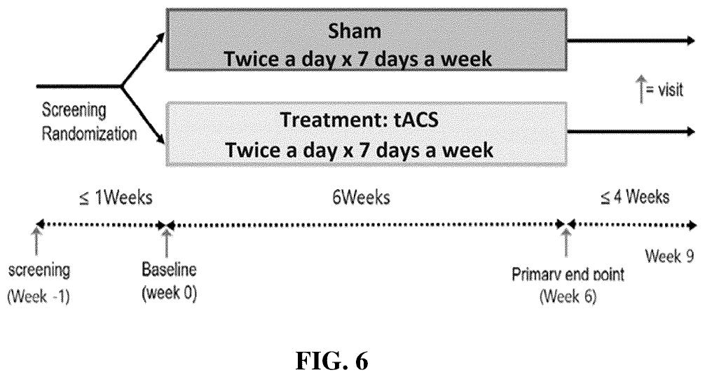

FIG. 6 is a diagram showing the clinical protocol of a clinical trial.

Referring to FIG. 6, clinical trials were conducted to confirm whether the brain stimulation device (100) could improve the patient's depression.

The purpose of the clinical trial is to verify the neurophysiological, molecular biological, anatomical, and behavioral effects of tACS stimulation on depression.

The subjects of the clinical trial were set as patients with anxiety/depressive symptoms aged 40 years or older and under 70 years.

The sample size for the clinical trial was set at a minimum of 60 people, considering a dropout rate of 15% in the exploratory clinical trial process, and the sample size for the confirmatory clinical trial was decided based on the exploratory pre-and post-comparison clinical results and the approval process of the Ministry of Food and Drug Safety.

The random allocation of the clinical trial was stratified by gender (female and male) and age (under or over 42 years) and was set to randomly mix two and four blocks so that the test group (the brain stimulation device of the present invention with applied parameters) and the control group (sham device with the same external and internal design as the brain stimulation device but without actual tACS-based combined stimulation) were assigned in a 1:1 ratio.

The efficacy evaluation indicators of the clinical trial were conducted with BDI-II and LEIDS-R to evaluate depression as the primary indicators, and the secondary indicators were conducted with blood tests, changes in HRV indicators such as SDNN and LF/HF, and the average amplitude and power of induced slow waves (theta, delta, gamma) brain waves.

The statistical analysis of the clinical trial used an Independent t-test or chi-square test and Paired t-test to determine the difference between the basic demographic variables and the baseline test results between the two groups (the average difference between the two groups was tested using the Independent t-test method, and if the observations of the two groups were paired, the paired t-test was applied).

As a result of the clinical trial, it was confirmed that the test group, which is the brain stimulation device (100) equipped with the tACS stimulation unit (313) of the present invention, had an effect on improving depression compared to the control group, which is the sham device.

### Effects of the Present Invention

The present invention can provide a personalized brain stimulation device capable of personalized brain stimulation that can improve the depressive state of the patient by determining the depressive state of the patient based on the biological information of the patient and delivering tACS (transcranial alternating current stimulation) based combined stimulation to the brain of the patient.

Specifically, the present invention can provide a personalized brain stimulation device capable of personalized brain stimulation that can improve the depressive state of the patient by determining the depression of the patient based on at least one of the biological information of the patient such as heart rate variability (HRV) information, electroencephalogram (EEG) information, heart rate information, stress information, body composition information, weight information, oxygen saturation information, pulse information, blood pressure information, iris information, voice information, vein information, and electrocardiogram (ECG) information and delivering tACS (transcranial Alternating Current Stimulation) based combined stimulation to the brain of the patient.

Further, the present invention can provide a personalized brain stimulation device capable of personalized brain stimulation that can enhance gamma-level brain waves in the brain waves of the patient through the entrainment of gamma oscillations when the depressive state of the patient is a major unipolar depression (MDD) state.

Moreover, the present invention can provide a personalized brain stimulation device capable of personalized brain stimulation that can reduce gamma-level brain waves in the brain waves of the patient through the entrainment of any one of delta, theta, alpha, and beta oscillations when the depressive state of the patient is a depressive disorder state caused by stress.

Furthermore, the present invention can provide a personalized brain stimulation device capable of personalized brain stimulation that can enhance gamma-level brain waves when the gamma level is lowest in the brain waves of the patient and reduce gamma-level brain waves when the gamma level is highest in the brain waves of the patient when the depressive state of the patient is a bipolar disorder (BD) state.

Meanwhile, the above-described embodiments of the present invention may be implemented through various means. For example, the embodiments of the present invention may be implemented through hardware, firmware, software, or a combination thereof.

In the case of hardware implementation, the methods according to the embodiments of the present invention may be implemented using one or more ASICs (Application Specific Integrated Circuits), DSPs (Digital Signal Processors), DSPDs (Digital Signal Processing Devices), PLDs (Programmable Logic Devices), FPGAs (Field

Programmable Gate Arrays), processors, controllers, microcontrollers, microprocessors, etc.

In the case of firmware or software implementation, the methods according to the embodiments of the present invention may be implemented in the form of modules, procedures, or functions that perform the functions or operations described above. The software code may be stored in a memory unit and driven by a processor. The memory unit may be located inside or outside the processor and may exchange data with the processor through various known means.

The detailed description of the preferred embodiments of the present invention as disclosed above is provided so that those skilled in the art can implement and practice the invention. While the present invention has been described with reference to the preferred embodiments, those skilled in the art will understand that the invention may be variously modified and changed without departing from the scope of the invention. For example, those skilled in the art can utilize the configurations described in the above embodiments in combination with each other. Therefore, the present invention is not intended to be limited to the embodiments shown herein but is to be accorded the broadest scope consistent with the principles and novel features disclosed herein.

The present invention may be embodied in other specific forms without departing from its spirit or essential characteristics. Therefore, the detailed description is to be considered as illustrative and not restrictive. The scope of the present invention should be determined by the reasonable interpretation of the appended claims, and all modifications within the equivalent range of the present invention are included in the scope of the present invention. The present invention is not intended to be limited to the embodiments shown herein but is to be accorded the broadest scope consistent with the principles and novel features disclosed herein. Furthermore, claims not explicitly cited in the claims may be combined to form embodiments or included as new claims by amendments after the application.

What is claimed is:

1. A personalized brain stimulation device comprising:
a sensor unit that measures biological information of an object;
a control unit that determines, based on the biological information of the object measured by the sensor unit, that the state of the object corresponds to a first state among a predetermined plurality of depression-related states; and
a stimulation unit that delivers a stimulus, which varies according to the first state determined by the control unit, to a brain of the object to entrain synchronized oscillation in multiple regions of the brain of the object,
wherein the stimulus is a first stimulus to entrain gamma oscillation synchronized in multiple regions of the brain, a second stimulus to entrain any one of delta, theta, alpha, and beta oscillation synchronized in multiple regions of the brain, or a third stimulus that combines the first stimulus and the second stimulus,
wherein the first, second, and third stimulus are an transcranial alternating current stimulation (tACS), and
wherein the transcranial alternating current stimulation switches on and off according to a predetermined first frequency, and is a first combined stimulus that applies the signal switched on according to the first frequency as stimulus according to a predetermined second frequency.

2. The personalized brain stimulation device according to claim 1, wherein the biological information comprises at least one of heart rate variability (HRV) information, electroencephalogram (EEG) information, heart rate information, stress information, body composition information, weight information, oxygen saturation information, pulse information, blood pressure information, iris information, voice information, vein information, and electrocardiogram (ECG) information.

3. The personalized brain stimulation device according to claim 1, wherein the predetermined plurality of depression-related states comprise a depressive disorder state caused by stress, a major unipolar depression (MDD) state, and a bipolar disorder (BD) state.

4. The personalized brain stimulation device according to claim 3, wherein the stimulation unit delivers the first stimulus to the brain when the first state determined by the control unit is the major unipolar depression state, and
wherein the delivery of the first stimulus enhances a gamma-level brain wave in a brain wave of the object through the entrainment of gamma oscillation.

5. The personalized brain stimulation device according to claim 4, wherein the stimulation unit delivers the second stimulus to the brain when the first state determined by the control unit is the depressive disorder state caused by stress, and
wherein the delivery of the second stimulus reduces the gamma-level brain wave in the brain wave of the object through the entrainment of any one of delta, theta, alpha, and beta oscillation.

6. The personalized brain stimulation device according to claim 5, wherein the stimulation unit delivers the third stimulus to the brain when the first state determined by the control unit is the bipolar disorder state,
wherein, when the first state of the object is the bipolar disorder state and the gamma-level brain wave in the brain wave of the object is the lowest, the first stimulus is delivered to the brain to enhance the gamma-level brain wave in the brain wave of the object, and
wherein, when the first state of the object is the bipolar disorder state and the gamma-level brain wave in the brain wave of the object is the highest, the second stimulus is delivered to the brain to reduce the gamma-level brain wave in the brain wave of the object.

7. The personalized brain stimulation device according to claim 2, wherein the brain wave information is the first brain wave obtained from the brain,
wherein the sensor unit measures the second brain wave of the object while the stimulus is being delivered to the brain, and
wherein the control unit determines whether a first response, which entrains synchronized oscillation in multiple regions of the brain of the object, is derived based on the second brain wave of the object measured by the sensor unit.

8. The personalized brain stimulation device according to claim 1, wherein the first frequency is applied to induce the entrainment of synchronized oscillation in multiple regions of the brain of the object,
wherein the second frequency is applied to induce membrane action potential and brain oscillation in multiple regions of the brain of the object, and
wherein the second frequency is higher than the first frequency.

9. The personalized brain stimulation device according to claim 8, wherein the control unit processes the first signal caused by the first combined stimulus as noise from the signals measured by the sensor unit, and
wherein the control unit determines whether the first response is derived based on the second signal excluding the first signal from the signals measured by the sensor unit.

10. The personalized brain stimulation device according to claim 9, wherein the control unit, when the first response according to the first combined stimulus is not derived, controls the stimulation unit to deliver a second combined stimulus to the brain of the object that at least one of the first frequency, the second frequency, and the output, the waveform and the period of the stimulus according to the second frequency is modified.

11. The personalized brain stimulation device according to claim 10, wherein the first response is related to the entrainment of gamma oscillation or any one of delta oscillation, theta oscillation, alpha oscillation and beta oscillation synchronized in multiple regions of the brain, and
wherein the first frequency is 30 Hz to 80 Hz to entrain the gamma oscillation synchronized in multiple regions of the brain of the object including the prefrontal cortex (PFC) and hippocampus of the object, 14 Hz to 29 Hz to entrain the beta oscillation, 8 Hz to 13 Hz to entrain the alpha oscillation, 4 Hz to 7 Hz to entrain the theta oscillation, or a frequency greater than 0 Hz and less than 4 Hz to entrain the delta oscillation.

12. The personalized brain stimulation device according to claim 11, wherein, based on the magnitude of the first signal being different from the magnitude of the second signal by at least a predetermined value, the signal interference caused by the first combined stimulus is negligible.
